(19) United States
(12) Patent Application Publication
(10) Pub. No.: US 2013/0197527 A1
(43) Pub. Date: Aug. 1, 2013

(54) SYSTEMS, METHODS AND DEVICES FOR TIBIAL RESECTION

(71) Applicants: Chris F. Scifert, Bartlett, TN (US); Stefan W. Kreuzer, Houston, TX (US)

(72) Inventors: Mark E. Nadzadi, Memphis, TN (US); Chris F. Scifert, Bartlett, TN (US); Stefan W. Kreuzer, Houston, TX (US)

(73) Assignee: Smith & Nephew, Inc., Memphis, TN (US)

(21) Appl. No.: 12/304,696

(22) PCT Filed: Jun. 12, 2007
(86) PCT No.: PCT/US07/71042

§ 371 Date: Sep. 5, 2012

(30) Foreign Application Priority Data
Jun. 12, 2006 (US) 60812849

(51) Int. Cl. A61B 17/17

(52) U.S. Cl. CPC A61B 17/1764
USPC 606/88

(57) ABSTRACT

Systems, methods and device for tibial resection comprise a first cutting guide and a second cutting guide. The first cutting guide (12) is configured to overlay a portion of the tibia and to direct a cutting instrument in a plane. The first cutting guide has a length extending from a generally medial portion of the tibia to a generally lateral portion of the tibia. The first cutting guide has a depth extending in a posterior direction generally perpendicular to the length and a groove extending along the length and depth of the first cutting guide such that the groove extends along a generally transverse plane. The second cutting guide (34) is oriented at an angle to the first cutting guide and configured to extend generally in a posterior direction from the first cutting guide (12). The second cutting guide limits the cutting instrument in the transverse plane from cutting bone.

### CROSS-REFERENCE TO RELATED APPLICATIONS

This application claims the benefit of U.S. Provisional Application No. 60/812,849 filed Jun. 12, 2006. The disclosure of this prior application is incorporated by reference in its entirety.

### BACKGROUND

1. Field of the Invention

The present invention relates to systems, methods, and devices for bone resection. More particularly, the present invention relates to systems, methods, and devices for tibial resections and soft tissue guided bone resections.

2. Related Art

Previous cutting instruments are all guided outside the bone, and once the instrument enters the bone it is no longer guided. Problems, such as tip deflection and skiving can affect bone cuts, implant alignment, and fixation. Previous instrumentation also will not fit under tight soft tissues, necessitating resection of those tissues or movement of those tissues.

### SUMMARY

A resection guide for a tibia comprises a first cutting guide and a second cutting guide. The first cutting guide is configured to overlay a portion of the tibia and to direct a cutting instrument in a plane. The first cutting guide has a length extending from a generally medial portion of the tibia to a generally lateral portion of the tibia. The first cutting guide has a depth extending in a posterior direction generally perpendicular to the length and a groove extending along the length and depth of the first cutting guide such that the groove extends along a generally transverse plane. The second cutting guide is oriented at an angle to the first cutting guide and configured to extend generally in a posterior direction from the first cutting guide. The second cutting guide limits the cutting instrument in the transverse plane from cutting bone.

Additionally, a resection guide may further comprise a third cutting guide configured to extend generally orthogonal to the transverse plane of the first cutting guide and oriented in the posterior direction of the second cutting guide.

Another embodiment may include a resection guide wherein the first and second cutting guides are configured to cut a medial portion of the tibia.

Additionally, a resection guide may further comprise a support structure. The support structure has a bone fixator configured to fixate the resection guide to the bone. The support structure may additionally include a cutting guide support configured to orient the varus/valgus angle of the first cutting guide.

Another embodiment may include a resection guide wherein the support structure has a port configured to receive a lateral resection guide.

One embodiment may include a resection guide wherein the lateral resection guide is fixed to the support structure with a connector.

Another embodiment may include a resection guide wherein the bone fixator is an extramedullary rod guide.

Additionally, the support structure may further comprise an offset configured to position the bone fixator away from the cutting guide support such that the bone fixator is outside the surgical field.

Additionally, the support structure may further comprise an offset configured to position the cutting guide support between the tibia and the patellar tendon and further position the bone fixator over the patellar tendon.

In one embodiment, the second cutting guide is a pin.

Additionally, the pin may include a cutout configured to retain the cutting instrument.

Another embodiment of the second cutting guide may include a sleeve having a cutout to retain the cutting instrument.

In another embodiment, the second cutting guide may extend into the tibia.

A method of resecting a portion of a tibia includes the step of orienting a first cutting plane of a first cutting guide in a transverse plane in a medial/lateral direction. The first cutting plane sets the varus/valgus angle of the cutting plane. Another step installs a second cutting guide in the first cutting plane. The second cutting surface extends distally in the first cutting plane and limits the range of motion of a cutting instrument in the direction of the medial/lateral resection. Another step cuts the tibia from the outer surface of the bone along the first cutting plane in the first cutting guide to the second cutting guide.

Another embodiment further comprises the step of orienting a third cutting guide generally perpendicular to the first cutting plane. The third cutting guide extends in a sagittal plane. Another step includes cutting the tibia with the cutting instrument from a proximal portion along the sagittal plane of the third cutting guide distally until the cutting instrument contacts the second cutting guide.

In another embodiment, the installing step may further comprise the step of drilling a hole through the bone such that the hole is aligned along the first cutting plane.

Additionally, the installing step may further comprise the step of inserting a pin into the bone.

Additionally, the second cutting guide may be a sleeve on a pin. The installing step further comprises the step of removing the pin from the sleeve when the sleeve is placed in the bone.

In one embodiment the method further comprises the step of attaching a lateral cutting guide to the first cutting guide after the cutting step.

In another embodiment the method further comprises the step of fixing the cutting guide to the bone.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is view of an embodiment of a tibial resection guide.

FIG. 2 is a view of a portion of the tibial resection guide of FIG. 1 placed on a tibia and receiving a cutting guide.

FIG. 3 is a view of the portion of the tibial resection guide of FIG. 2 with the cutting guide installed.

FIG. 4 is a view of an embodiment of a tibial resection guide for a medial resection.

FIG. 5 is a view of an embodiment of a lateral resection guide oriented relative to a medial resection.

FIG. 6 is another view of the embodiment of the lateral resection guide of FIG. 5.

FIG. 7 is a view of an embodiment of a portion of tibial resection guide for a medial resection.

FIG. 8 is another view of the embodiment of the tibial resection guide of FIG. 7.

FIG. 9 is a view of other portions of the tibial resection guide of FIG. 7.

FIG. 10 a view of an embodiment of a tibial resection guide.

FIG. 11 is an end view of an embodiment of a pin having a cutout portion.

FIG. 12 is an end view of another embodiment of a pin having a cutout portion.

FIG. 12 is an end view of another embodiment of a pin having a cutout portion.

### DETAILED DESCRIPTION OF THE EMBODIMENTS

The following description of the preferred embodiment(s) is merely exemplary in nature and is in no way intended to limit the invention, its application, or uses.

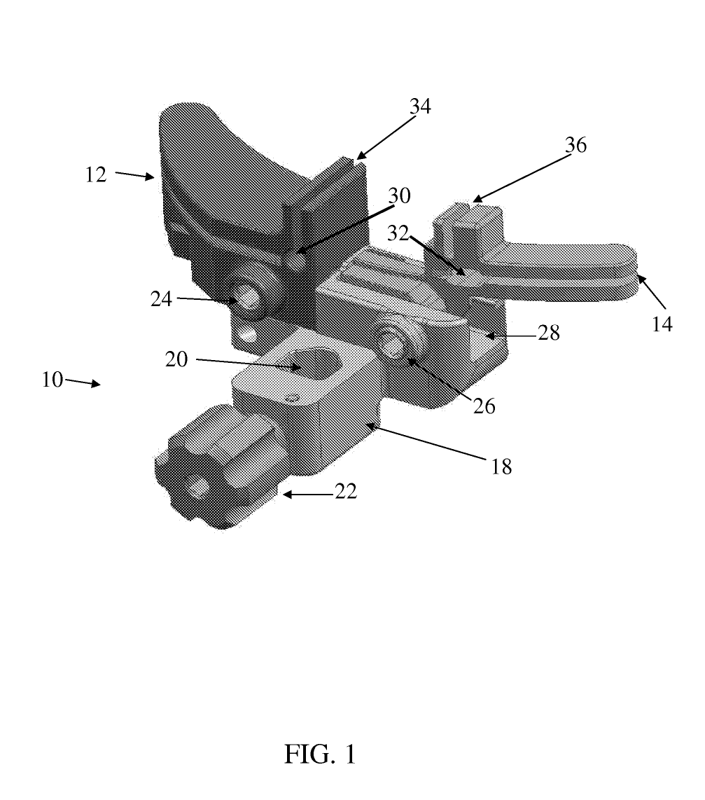

Turning to the drawing figures, FIG. 1 is view of an embodiment of a tibial resection guide 10. The guide 10 includes a medial cutting guide 12, a lateral cutting guide 14 and a bone fixator 18. The bone fixator 18, in this embodiment, is an extramedullary (EM) rod connector having an extramedullary rod guide 20 and a tightening knob 22. The medial and lateral cutting guides 12 and 14 include set screws 24 and 26 which lock the medial and lateral cutting guides 12 and 14 in place along a variable medial/lateral slots (slot 28 for the lateral side). Pin slots 30 and 32 in the cutting guides 12 and 14 are configured to receive cutting guides. Vertical cutting guides 34 and 36 are oriented above the pin slots 30 and 32.

The bone fixator 18 is configured to fix the cutting guide to the bone. The EM rod guide may be oriented to account for varus/valgus angle for the knee. Additionally, fixation pins may be used though openings in the resection guide 10 to fix the resection guide 10 to the bone. When the resection guide 10 is properly aligned and oriented on the EM rod, then the knob 22 may be tightened to fix the resection guide 10 in place.

The cutting guides 12 and 14 are oriented with respect to the bone fixator 18 to align the cutting surfaces for the medial and lateral portions of the tibia. The set screws 24 and 26 set the medial and lateral cutting guides 12 and 14 in place in the medial/lateral direction. When the set screws 24 and 26 are loosened, then medial and lateral cutting guides may be variably positioned laterally and medially. Additionally, the set screws 24 and 26 may be removed to allow for the cutting guides 12 and 14 to be individually removed. The cutting guides 12 and 14 may be used independently, then, to minimize the size of the resection guide 10. A smaller resection guide 10 may help to minimize the incision size and minimize soft tissue resections or displacements.

The cutting guides 12 and 14 include horizontal cutting planes and vertical cutting planes. The horizontal and vertical cutting planes define the horizontal and vertical cutting surfaces for the tibia. When these guides 12 and 14 are used, the tibia will have medial and lateral resections with a shelf maintaining natural bone for the medial condyle between the resections where soft tissue may be maintained. For example, posterior and anterior cruciate ligaments attach to the tibia along the medial condyle of the tibia, and may be saved when the medial and lateral compartments are individually cut.

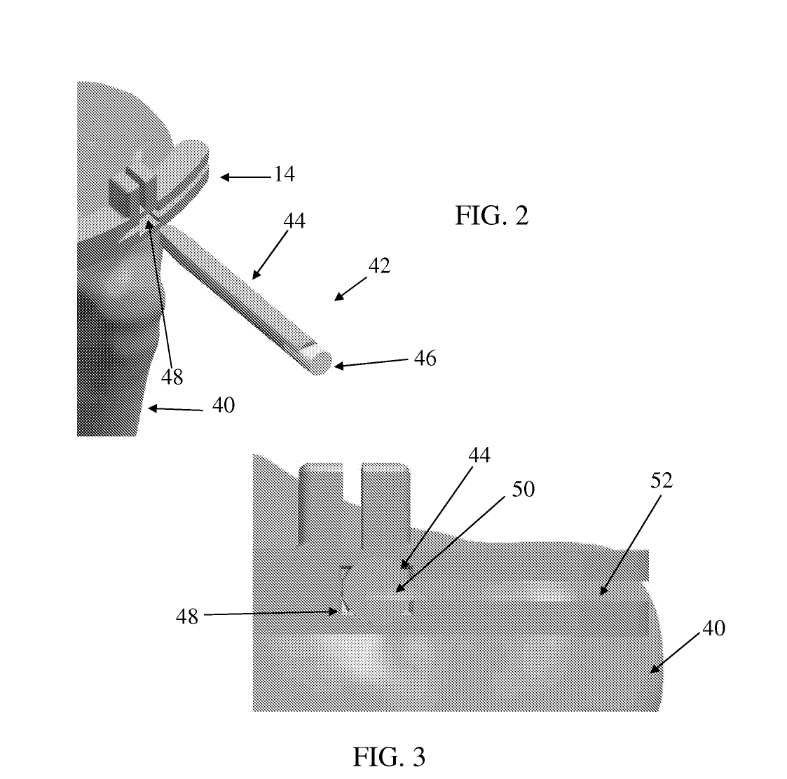

In operation, the guide 10 is placed on an EM rod and fixed to the rod. The angle of the rod fixes the varus/ valgus rotation of the resection guide 10. Generally, the medial compartment is resected first. The medial cutting guide 12 is positioned on the tibia. A pin (shown, for example, in FIG. 2) is inserted into the tibia. The horizontal cut in a transverse plane to the tibia is cut. The pin is set along the transverse plane. The vertical cutting guide 34 is positioned at an angle to the transverse plane and extends to the pin. Thus, the pin is in the transverse plane formed by the horizontal cutting guide and the sagittal plane of the vertical cutting guide. The cut in the transverse plane is cut to the pin, and the sagittal plane is cut down from above the tibia to the pin.

The pin cutting guide limits the cuts in both the transverse plane and sagittal plane. This minimizes the possibility of undercutting the resection. This also protects from overextending the cuts which may damage soft tissue. By creating a physical stop in the path of the cuts, the cuts may not extend past the stops. The pin may also provide a fillet at the corner to reduce stress risers in the bone. Additionally, as discussed below, the pin may limit tip deflection and better align or stabilize the cutting instruments during the cuts.

Turning now to FIG. 2, FIG. 2 is a view of a portion of the tibial resection guide 10 of FIG. 1 placed on a tibia 40 and receiving an anterior/posterior cutting guide 42. The lateral cutting guide 14 is positioned against a lateral portion of the tibia 40. The anterior/posterior cutting guide 42 may extend anterior/ posterior into the tibia and includes an outer sheath 44 and a pin 46. The anterior/posterior cutting guide 42 is inserted into a receiving slot 48 in the transverse guide of the lateral cutting guide 14. The anterior/posterior cutting guide 42 may be driven into the bone. Alternatively, a hole may be drilled into the bone and the anterior/posterior cutting guide 42 may be inserted into the receiving slot 48. Once the anterior/posterior cutting guide 42 is positioned within the receiving slot 48, the pin 46 may be removed from the sheath 44 leaving a cutting slot, as shown in FIG. 3.

Turning now to FIG. 3, FIG. 3 is a view of the portion of the tibial resection guide of FIG. 2 with the cutting guide 42 installed. The pin of the anterior posterior cutting guide 42 has been removed leaving the sheath 44 in the receiving slot 48. An A/P sheath slot 50 extends from the anterior portion of the tibia 40 to the posterior portion of the tibia 40. The sheath slot 50 allows for a cutting instrument to be inserted along the sheath slot 50 to start the cut and control the tip of the cutting instrument. The cut continues within a horizontal slot 52 to the lateral side of the tibia. The sheath slot 50 also stabilizes the transverse cut and properly aligns the transverse cut. As the transverse cut is made, the cut may not extend more medially than the sheath slot 50.

A vertical slot 54 extends from a proximal portion of the tibia 40 to the sheath 44. In this embodiment, the sheath is oriented as a stop and does not create a starting point inferiorly for the vertical cut. However, the receiving slot 48 and the sheath slot 50 may be oriented so that the vertical slot 54 may be aligned with the sheath slot 50.

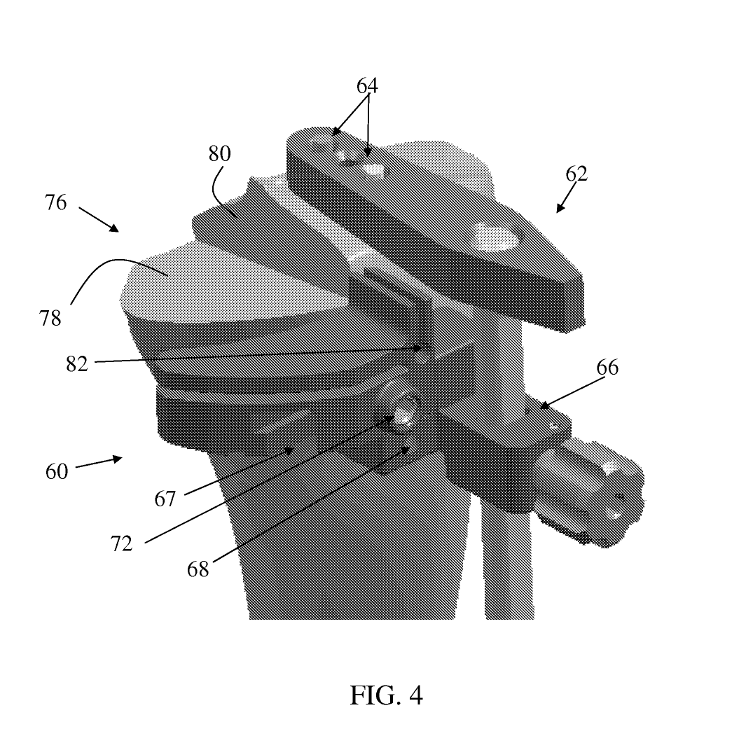

Turning now to FIG. 4, FIG. 4 is a view of an embodiment of a tibial resection guide 60 for a medial resection. The tibial resection guide 60 is connected to the bone with a spike rod 62. The spike rod 62 attaches to an EM guide 66 of the tibial resection guide 60. Pins 64 attach the medial cutting guide to the bone. Additional bone fixation may be achieved with a pin through a pinhole 68. A variable M/L slot 67 allows for the medial guide 60 to be moved medially and laterally with respect to the tibia, and is thus similar to the slot 28 of FIG. 1. A set screw 72 fixes the M/L position of the cutting guide 60.

The pins 64 may be of different heights so that a first pin may be hammered into the tibia first, and then the spike rod (and thus the EM rod guide 66 and cutting guide 60) may be rotated before the second, shorter pin 64 is knocked into the bone. Once the second shorter pin is placed, the guide 60 is fixed to the bone.

A medial cutout 76 shows the transverse cut 78 and the vertical cut 80 of the tibia. An A/P pin slot 82 is positioned so that an A/P pin would be placed at the intersection of the transverse cut 78 and the vertical cut 80 so that when the transverse and vertical cuts are made, the A/P pin would limit the lateral edge of the transverse cut and the inferior edge of the vertical cut.

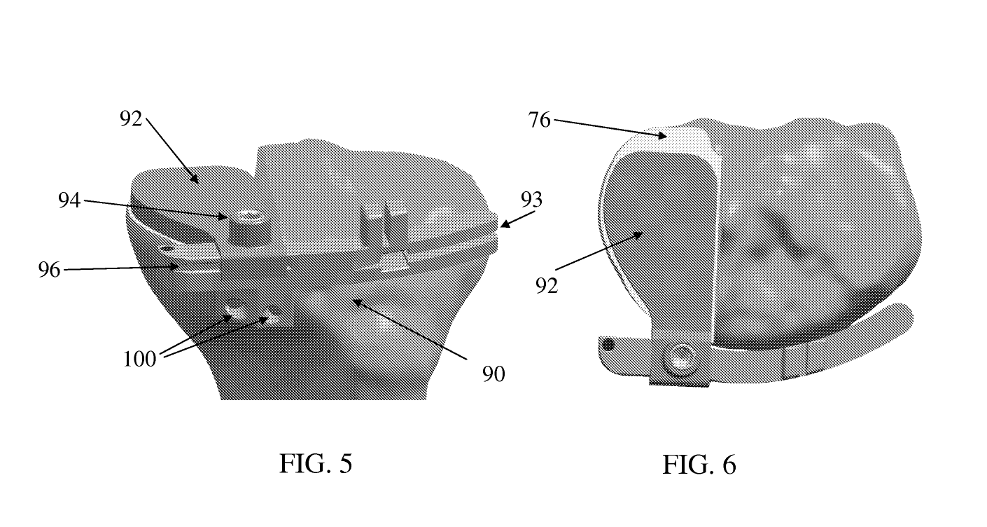

Turning now to FIG. 5, FIG. 5 is a view of an embodiment of a lateral resection guide 90 oriented relative to a medial resection. For example, the lateral resection guide 90 may be used with the medial resection guide of FIG. 4. The lateral guide 90 includes a medial pad 92 which orients a lateral cutting guide 93 to the tibia. A set screw 94 sets the medial lateral position of the lateral resection guide 90 in a slide 96. Fixation is achieved by extending the knee and having the medial condyle of the femur pressing down on the medial pad 92. Additionally, pinholes 100 may be used to fixate the lateral resection guide 90 to the bone. Extending the femur also relaxes the patella, moving the patella out of the way for lateral cuts. The horizontal and vertical cutting guides for the lateral resections are operated similar to the cuts discussed previously.

The cuts may be formed either using a sagittal saw or a reciprocating saw. When using a reciprocating saw, then the preferred cut starts at the A/P guide and progresses toward the posterior of the tibia within the slot of the A/P guide. After completing a cut from the anterior to the posterior of the tibia, then the blade is pushed out within the transverse slot of the lateral guide 94.

Turning now to FIG. 6, FIG. 6 is another view of the embodiment of the lateral resection guide of FIG. 5. The medial pad 92 sits on the medial tibial cutout 76. Any variation in the medial cut 76 will be transferred to the lateral side through the medial pad 92. Thus, misalignment between the medial and lateral cuts is minimized through the sequential cutting of first the medial than the lateral compartments.

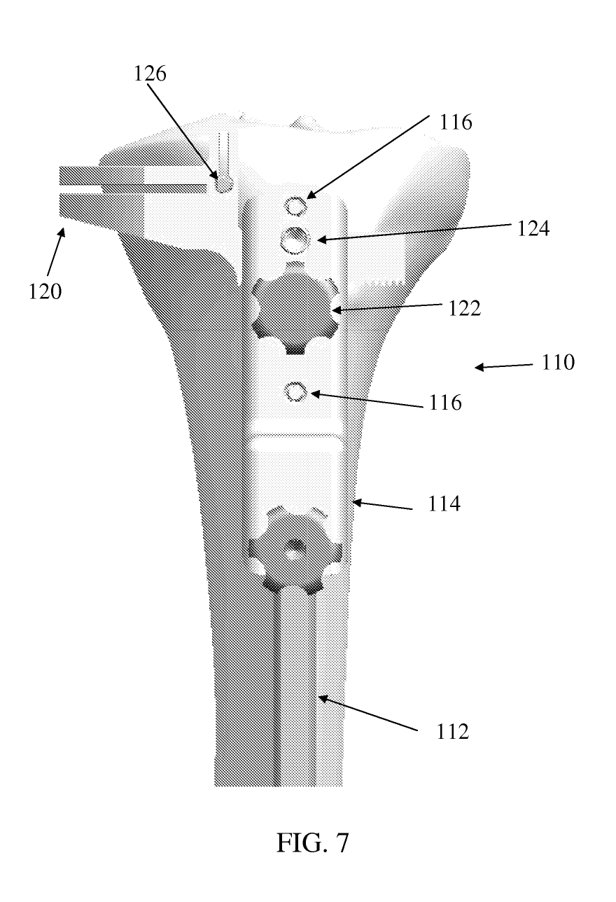

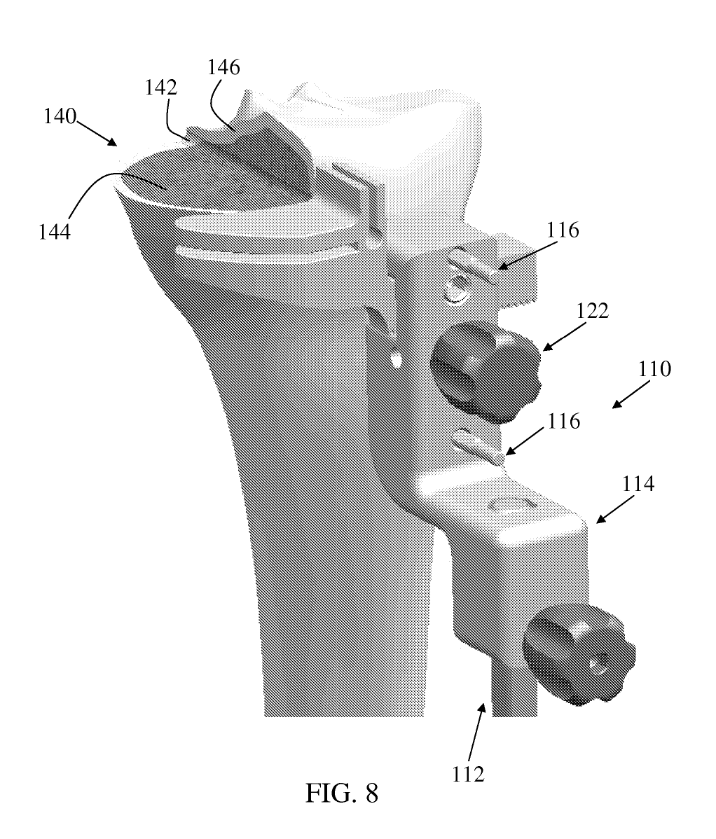

Turning now to FIGS. 7 and 8, FIG. 7 is a view of an embodiment of a portion of tibial resection guide 110 for a medial resection. An extra medullary rod 112 fixates the resection guide 110 with a step down block 114. Pins 116 further fixate the guide 110 to the bone. A medial cutting guide 120 may be moved medially/laterally with a knob 122 and set in place by a set screw 124. An A/P cutting guide 126 through the medial cutting guide 120 limits the cuts of a medial cutout. FIG. 8 is another view of the embodiment of the tibial resection guide 110 of FIG. 7. A medial cutout 140 shows a filleted cut surface 142 at the intersection of a transverse cut 144 and a vertical cut 146

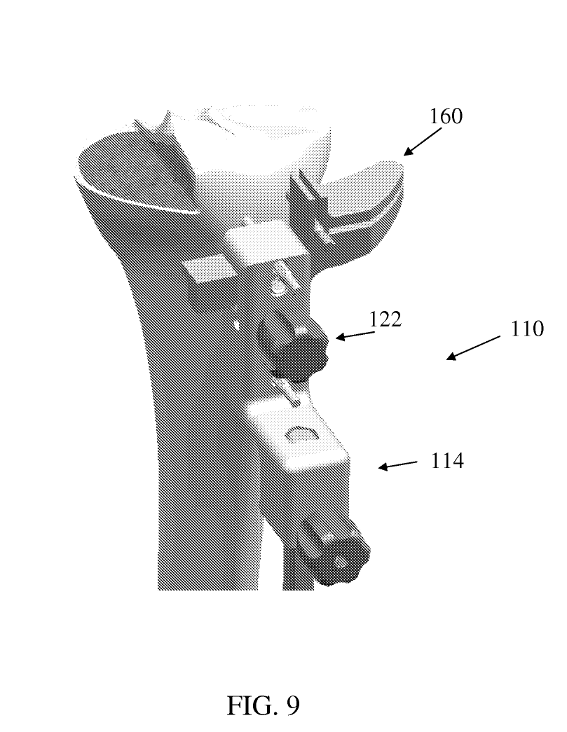

Turning now to FIG. 9, FIG. 9 is a view of other portions of the tibial resection guide 110 of FIG. 7. A lateral cutting guide 160 is received in the same slot as the medial guide of FIGS. 7 and 8. The knob 122 may orient the lateral cutting guide 160 in the medial/ lateral direction. Because the resection guide 110 is fixed in the same orientation as when the medial cuts were made, the relative orientation of the medial cuts to the lateral cuts are consistent.

The step down block 114 is an offset that moves part of the bone fixation and the part that orients the guide 110 away from the cutting planes and cutting surfaces. This allows for more access and added views of the cutting planes.

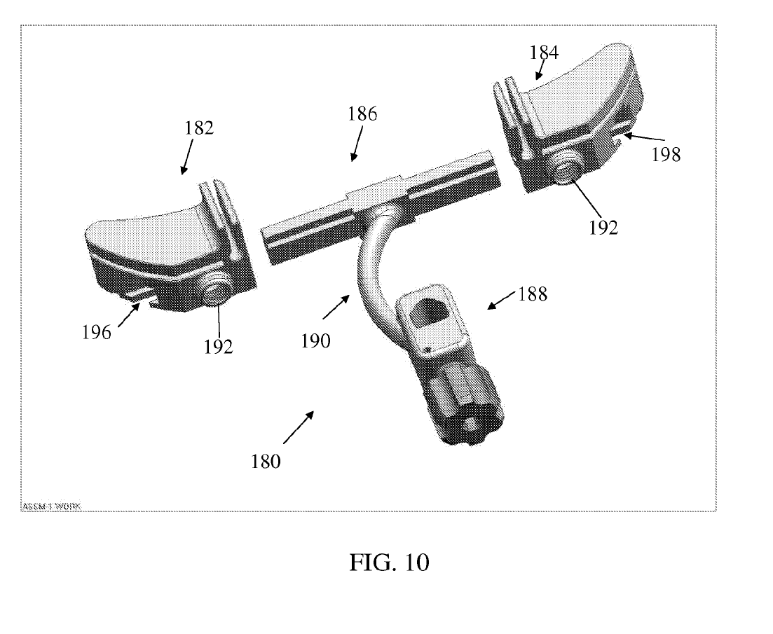

Turning now to FIG. 10, FIG. 10 a view of an embodiment of a tibial resection guide 180. A medial cutting guide 182 and a lateral cutting guide 184 are received on a cutting guide support. In this embodiment, the cutting guide support is a guide post 186. A bone fixator 188 is connected to the post 186 by an offset 190. Set screws 192 fix the cutting guides 182 and 184 to the post 186. The post 186 allows for medial/lateral movement of the cutting guides 182 and 184 along tracks 196 and 198 respectively.

The tibial resection guide 180 is generally smaller in size than conventional resection guides. The offset 190 is shaped to allow for the post 186 to be inserted within an incision while the bone fixator 188 remains outside the incision. The curve in the offset 190 positions the post 186 between the tibia and the patellar tendon and position the bone fixator 188 over the patellar tendon. In order to minimize the size of the resection guide 180, the cutting guides 182 and 184 may be used individually.

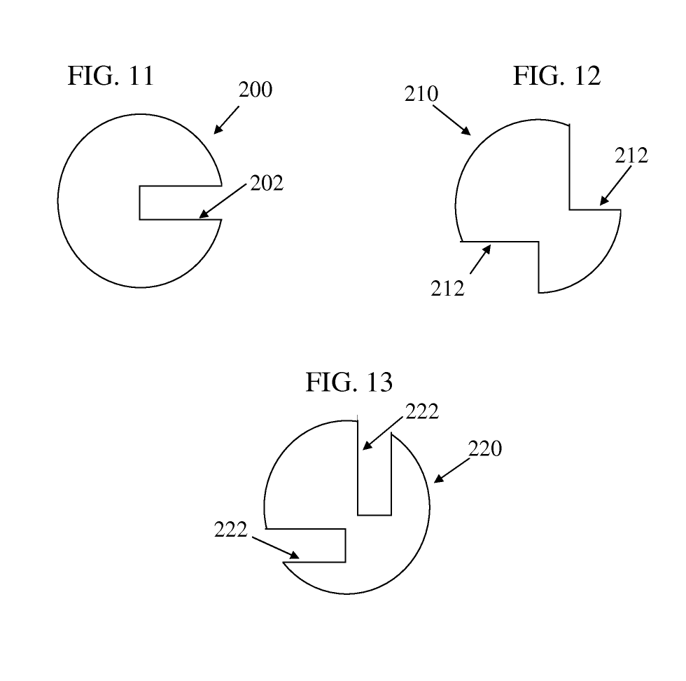

Turning now to FIGS. 11-13, FIG. 11 is an end view of an embodiment of a pin 200 having a cutout portion 202. FIG. 12 is an end view of another embodiment of a pin 210 having a cutout portions 212. FIG. 12 is an end view of another embodiment of a pin 220 having a cutout portions 222. Each of these pins 200, 210, and 220 may be used as pins with the embodiments of resection guides previously discussed. The cutout portion 202 is a portion that is limited to a single slot in the A/P direction of the pin 200. The cutout portions 212 and 222 are configured to capture a cutting instrument in orthogonal planes in the A/P direction. While cutout portions 212 extend tangential to the cross section of the pin 210, the cutout portions 222 extend along secants of the cross section of the pin 220. While the cutouts 212 and 222 are positioned orthogonally, the cutouts 212 and 222 may be positioned at any angle relative to one another. Additionally, sheaths may be used with the pins to fill the cutouts initially if the pins are driven into the bone instead of placed within pre-drilled holes.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A resection guide for a tibia, comprising:
a. a first cutting guide configured to overlay a portion of the tibia and to direct a cutting instrument in a plane, the first cutting guide having a length extending from a generally medial portion of the tibia to a generally lateral portion of the tibia, the first cutting guide having a depth extending in a posterior direction generally perpendicular to the length, the first cutting guide having a groove extending along the length and depth of the first cutting guide such that the groove extends along a generally transverse plane;
b. a second cutting guide oriented at an angle to the first cutting guide and configured to extend generally in a posterior direction from the first cutting guide such that the second cutting guide limits the cutting instrument in the transverse plane from cutting bone.

2-9. (canceled)

10. The resection guide of claim 1, wherein the second cutting guide is a pin.

11. The resection guide of claim 10, wherein the pin has a cutout configured to retain the cutting instrument.

12. The resection guide of claim 1 wherein the second cutting guide is a sleeve having a cutout to retain the cutting instrument.

13. The resection guide of claim 1 wherein the second cutting guide extends into the tibia.

14-20. (canceled)

21. A tibial resection guide, comprising:
a first cutting guide configured to overlay an anterior portion of the tibia, the first cutting guide having at least one guide slot arranged along a cutting plane, the guide slot extending through the first cutting guide in an anterior-posterior direction and dimensioned to direct displacement of a cutting instrument generally along the cutting plane; and
a second cutting guide comprising an elongate member extending from a posterior end of the first cutting guide in the anterior-posterior direction and positioned within the cutting plane with the guide slot extending to the elongate member, the elongate member defining a stop structured and positioned to limit displacement of the cutting instrument along the cutting plane.

22. The resection guide of claim 21, wherein the at least one guide slot comprises:
a first guide slot arranged along a first cutting plane; and
a second guide slot arranged along a second cutting plane transverse to the first cutting plane; and
wherein the elongate member is positioned within both the first cutting plane and the second cutting plane.

23. The resection guide of claim 22, wherein each of the first and second guide slots extends to the elongate member; and
wherein the stop defined by the elongate member is structured and positioned to limit displacement of the cutting instrument along both the first cutting plane and the second cutting plane.

24. The resection guide of claim 21, wherein the first cutting guide includes a pin receiving opening extending through the first cutting guide in the anterior-posterior direction and communicating with the guide slot; and
wherein the second cutting guide comprises an elongate pin positioned within the pin receiving opening of the first cutting guide and with an anterior portion of the elongate pin positioned in communication with the guide slot.

25. The resection guide of claim 24, wherein the elongate pin defines a cut out region extending along a length of the elongate pin in the anterior-posterior direction, the cut out region generally aligned with the guide slot of the first cutting guide and dimensioned to receive the cutting instrument therein.

26. The resection guide of claim 21, wherein the second cutting guide is removably positioned within the guide slot.

27. The resection guide of claim 26, wherein the second cutting guide comprises an elongate pin including an anterior portion positioned in communication with the guide slot.

28. The resection guide of claim 26, wherein the second cutting guide comprises an inner pin removably positioned within an outer sleeve.

29. The resection guide of claim 21, wherein the elongate member defines at least one cut out region extending along a length of the elongate member in the anterior-posterior direction, the at least one cut out region generally aligned with the guide slot of the first cutting guide and dimensioned to receive the cutting instrument therein.

30. The resection guide of claim 29, wherein the second cutting guide comprises an inner pin removably positioned within an outer sleeve, and wherein the outer sleeve defines the at least one cut out region.

31. The resection guide of claim 29, wherein the at least one cut out region of the elongate member communicates with the guide slot of the first cutting guide.

32. The resection guide of claim 29, wherein the first cutting guide includes a pin receiving opening extending through the first cutting guide in the anterior-posterior direction and communicating with the guide slot; and
wherein the elongate member comprises a pin positioned within the pin receiving opening of the first cutting guide, the pin defining the at least one cut out region positioned in communication with the guide slot.

33. The resection guide of claim 29, wherein the at least one guide slot comprises:
a first guide slot arranged along a first cutting plane; and
a second guide slot arranged along a second cutting plane transverse to the first cutting plane; and
wherein the at least one cut out region includes:
a first cut out region generally aligned with the first guide slot; and
a second cut out region generally aligned with the second guide slot; and
wherein each of the first and second cut out regions are dimensioned to receive the cutting instrument therein.

34. The resection guide of claim 21, wherein the cutting instrument includes a cutting blade positioned within the guide slot and guidingly displaced generally along the cutting plane; and
wherein the cutting blade engages the stop defined by the elongate member of the second cutting guide to limit displacement of the cutting blade along the cutting plane.

35. A tibial resection guide, comprising:
a first cutting guide configured to overlay an anterior portion of the tibia, the first cutting guide having a first guide slot arranged along a first cutting plane and a second guide slot arranged along a second cutting plane generally orthogonal to the first cutting plane, each of the first and second guide slots extending through the first cutting guide in an anterior-posterior direction and dimensioned to direct displacement of a cutting instrument generally along the first and second cutting planes, respectively; and
a second cutting guide comprising an elongate member extending from a posterior end of the first cutting guide in the anterior-posterior direction and positioned within both the first and second cutting planes with at least one of the first and second guide slots extending to the elongate member, the elongate member defining a stop structured and positioned to limit displacement of the cutting instrument along at least one of the first and second the cutting planes.

36. The resection guide of claim 35, wherein each of the first and second guide slots extends to the elongate member.

37. The resection guide of claim 35, wherein the cutting instrument includes a cutting blade positioned within one of the first and second guide slots and guided generally along a respective one of the first and second cutting planes; and
wherein the cutting blade engages the stop defined by the elongate member of the second cutting guide to limit displacement of the cutting blade along the respective one of the first and second cutting planes.

38. A tibial resection guide, comprising:
a first cutting guide configured to overlay an anterior portion of the tibia, the first cutting guide having at least one guide slot arranged along a cutting plane, the guide slot extending through the first cutting guide in an anterior-posterior direction and dimensioned to direct displacement of a cutting instrument generally along the cutting plane; and
a second cutting guide comprising an elongate member extending from a posterior end of the first cutting guide in the anterior-posterior direction and positioned within the cutting plane, the elongate member defining at least one cut out region extending along a length of the elongate member in the anterior-posterior direction, the cut out region generally aligned with the guide slot of the first cutting guide and dimensioned to receive the cutting instrument therein.

39. The resection guide of claim 38, wherein the guide slot of the first cutting guide extends to the elongate member.

40. The resection guide of claim 39, wherein the cut out region of the elongate member communicates with the guide slot of the first cutting guide.

41. The resection guide of claim 38, wherein the first cutting guide includes a pin receiving opening extending through the first cutting guide in the anterior-posterior direction and communicating with the guide slot; and
wherein the elongate member comprises an elongate pin positioned within the pin receiving opening of the first cutting guide, the elongate pin defining the cut out region positioned in communication with the guide slot.

42. The resection guide of claim 41, wherein the elongate pin comprises an inner pin removably positioned within an outer sleeve, and wherein the outer sleeve defines the cut out region positioned in communication with the guide slot.

43. The resection guide of claim 38, wherein the at least one guide slot comprises:
a first guide slot arranged along a first cutting plane; and
a second guide slot arranged along a second cutting plane transverse to the first cutting plane; and
wherein the elongate member is positioned within both the first cutting plane and the second cutting plane.

44. The resection guide of claim 43, wherein the at least cut out region comprises:
a first cut out region generally aligned with the first guide slot; and
a second cut out region generally aligned with the second guide slot; and
wherein each of the first and second cut out regions are dimensioned to receive the cutting instrument therein.

45. The resection guide of claim 44, wherein the second cutting guide comprises an inner pin removably positioned within an outer sleeve, and wherein the outer sleeve defines the first and second cut out regions.
